(19) United States
(12) Patent Application Publication
(10) Pub. No.: US 2007/0016984 A1
(43) Pub. Date: Jan. 18, 2007

(54) Low pungency, long day onion

(76) Inventor: Scott Hendricks, Sun Prairie, WI (US)

Correspondence Address: ARNOLD & PORTER LLP;ATTN: IP DOCKETING DEPT., 555 TWELFTH STREET, N.W., WASHINGTON, DC, 20004-1206, US

(21) Appl. No.: 11/183,202

(22) Filed: Jul. 15, 2005

(51) Int. Cl. A01H 5/00

(52) U.S. Cl. 800/295; 800/321

(57) ABSTRACT

A low pungency, long-storing long day onion plant.

### BACKGROUND OF THE INVENTION

1. Field of the Invention

The present invention relates to plant varietal development, particularly the development of a low pungency, long day length type onion.

2. Description of Related Art

Onions belong to the lily family, Amaryllidaceae, and the genus, Allium. Alliums comprise a group of perennial herbs having bulbous, onion-scented underground leaves, including such commonly cultivated crops as garlic, chives, shallots and leeks. It also includes ornamental species grown for their flowers.

Onions are an important vegetable world-wide, ranking second among all vegetables in economic importance with an estimated value of $6 billion dollars annually. The onion is also one of the oldest cultivated vegetables in history. The common garden onions are in the species Allium cepa. Onions are classified in numerous ways, by basic use, flavor, color, shape of the bulb, and day length. Onions come in white, yellow, and red colors. The bulb may be rounded, flattened, or torpedo shaped.

Commercial onions include “storage onions”, “fresh onions”, “pearl or mini onions”, and “green onions”. “Fresh onions” tend to have a lighter color with a thin skin, a milder, sweeter flavor, and must be eaten fresh as they do not keep well. These onions are available in red, yellow, and white colors, and are often sold under the name of their region, e.g., Sweet Imperials, Vidalias, Walla Walla Sweets and Texas Sweets. Perhaps the best known of the fresh onions is the Bermuda onion. Fresh onions are available beginning in March or early April and can be purchased until August.

Storage onions are available from harvest, which is at the beginning of August, and are stored and available throughout the winter months up to about March. Storage onions have a darker skin that is thicker than that of a fresh onion. They are also known for intense, pungent flavor, higher percentage of solids and desirable cooking characteristics. These onions are also available in red, yellow and white colors. Not all long day length type (long day type) onions are suitable for storage. A true storage onion is one that can be harvested in late summer or fall, and stored, under proper conditions, until the spring, when the fresh onion crop is again available.

“Spanish onion”, “Spanish onions”, or “Spanish type” are terms applied to various long-day onions, generally yellow, though some white, and generally varieties that are large and globe-shaped. Spanish onion is commonly applied to various long day type onions of the type grown in western states of the United States (California, Idaho, Oregon, Washington, Colorado) with a bulb size averaging 300-700 grams (g) (typically over 3 inches up to 4 inches but also up to 5 inches in diameter for bulbs classified as “colossal”).

Onion varieties initiate bulbing when both the temperature and a minimum number of daylight hours reach certain levels. When onions are first planted, they initially develop their vegetative growth, with no sign of bulb formation until the proper day length for that onion variety triggers the signal to the plant to stop producing above ground vegetative growth and start forming a bulb. Onions are thus sensitive to the hours of daylight and darkness they receive, and for most varieties it is only when the specific combination of daylight and darkness is reached, that the bulb starts to form. Onions are therefore classified by the degree of day length that will initiate bulb formation. Onions are described as short-, intermediate-, and long-day length types. Short day means that bulbing will initiate at 11 to 12 hours of daylight. Intermediate day is used for onions bulbing at 12 to 14 hours of daylight. Long day means onions require 14 or more hours of daylight for bulb formation to start.

Growers producing onions in more northerly climates plant long-day length onions. Daylight length varies greatly with latitude, and at higher latitudes long-day onions will produce sufficient top growth before the day length triggers bulbing to produce a large bulb. A short-day onion grown in the North will bulb too early and produce relatively small bulbs.

Short day onions are preferred for southern areas such as southern Texas, southern California and Mexico. If a long day type onion is planted in such a short day climate, it may never experience enough day length to trigger the bulbing process.

Onions are also classified on flavor, with the common designations of sweet, mild, and pungent. The flavor of the onion is a result of both the type of onion and the growing conditions. For instance, soils containing a high amount of sulfur grow more pungent flavored onions. Sweetness in onions is caused by the sugars glucose, fructose and sucrose. Onions also contain polymers of fructose called fructans. Onion cultivars differ quite markedly in the relative amounts of sucrose, glucose, fructose and fructans which they contain. They also differ in sugars according to length of storage and location in the bulb. Short day cultivars, which are poor storers, tend to have higher levels of sucrose, fructose and glucose, but hardly any of the fructans. In contrast, long day type cultivars and intermediate storage cultivars such as Pukekohe Longkeeper have less sucrose, glucose and fructose and higher amounts of fructans.

The fructans do not play a role in sweetness. The balance between levels of pungency and levels of sugars determines the perception of pungency in an onion. High levels of pungency can mask high levels of sugars so that the onion is not perceived as sweet. Onions with low levels of pungency but low levels of sugars can be perceived as bland. Ideally a low pungency onion would have high levels of sugars and lower levels of pungency.

It is believed that sunlight strongly influences the development of pungent flavors. While compounds such as sugars and organic acids contribute to the flavor of onions, it is a special class of biologically active organosulfur compounds which give onions their distinctive flavor and aroma. Pungency in onions is caused by these volatile sulfur compounds, some of which affect the eyes when onions are first cut and induce tearing (often called lachrymatory effect). There are 3 different flavorprecursors in onions: 1-propenyl cysteine sulfoxide, which is usually found in the highest concentration; methyl-cysteine sulfoxide, which is normally found in lesser concentration; and propyl cysteine sulfoxide, which is found in the lowest concentration.

Storage conditions are also known to affect pungency, and though the research is conflicting, most studies show an increase in pungency for most long day type onions during storage. See Shock, C. C., E. B. G. Feibert, and L. D. Saunders. 2004. Pungency of Selected Onion Varieties Before and After Storage. Oregon State University, Malheur Experiment Station Special Report 1055: 45-46.

Within intact cells the enzyme alliinase is compartmentalized in the cell vacuole and the flavor precursors are found in the cytoplasm. A reaction, therefore, only occurs when onion tissues are damaged and the enzyme and substrate are brought together as organelles are disrupted. The kinetics of decomposition are different for each specific flavor precursor. The decomposition of 1-propenyl cysteine sulfoxide is almost instantaneous, while the methyl and propyl cysteine sulfoxide decomposition occurs in several minutes. Primary products produced from flavor precursor decomposition include pyruvate, ammonia and chemically unstable sulfenic acids. Among the sulfenic acids is the lachrymator, or tear producing compound, characteristic of onions. The sulfenic acids undergo further rearrangement to form thiosulfinates, which are responsible for the characteristic flavor of onions.

Flavor precursor formation begins with the uptake of sulfate (SO₄⁻²) by the onion, its reduction to sulfide, and subsequent assimilation into cysteine by light-dependent reactions in the leaves of the plant. Glutathione, a tripeptide of cysteine is then synthesized. This the starting point of the flavor precursor biosynthetic pathway. The pathways leading to the synthesis of each flavor precursor are not fully understood, although sulfur is known to be transformed through several identifiable peptide intermediates, each unique to a specific flavor precursor.

Researchers have recently developed a tool for documenting differences among onion flavor using a laboratory analysis of pyruvic acid development (PAD). Pyruvic acid has been shown to correlate well with flavor consumer flavor perception. The PAD measurements are gaining acceptance within the industry as a clearer index of onion mildness, even though pungency is assessed solely by the amount of enzymatic pyruvic acid. For most commercial onions, pyruvic acid levels fall between about 1 and about 18 micromoles per a gram fresh weight. PAD units are given in micromoles pyruvic acid per gram, fresh weight (μM/g FW). Short day onions marketed as low pungency onions will typically have PAD values of 5.5 μM/g FW or less. Onion bulbs having a PAD of 5.5 μM/g FW or less are considered sweet according to Vidalia Labs sweet onion certification specifications (Shock, C. C., E. B. G. Feibert, and L. D. Saunders. 2004. Onion Production from Transplants in the Treasure Valley. Oregon State University, Malheur Experiment Station Special Report 1055: 47-52). There are on-going discussions within the industry about the actual limits for sale as a low pungency onion, and some producers or shippers would prefer to set the upper limit at 4.0 μM/g FW. But, it is still common to see onions which are successfully marketed as low pungency onions with PADs of 7 and even above.

As a reference, Vidalia onions (a common industry standard for sweet onions) grown under low sulfur conditions are almost always in the low pungency category, Pukekohe Longkeeper (Creamgold) onions are usually about 7 μM/g FW and above and white dehydration onions are generally in the 15 μM/g FW range. Even for the Vidalia onion, PADs above 4.0 in individual onions are not uncommon.

Long day onions are generally grown in the northern states, because of their requirement for long days to initiate bulb production. For this reason, long day type storage varieties do extremely well in the northern states of the United States and Canada, regions that have the required 14-16 hours of day-length during the summer. There are no long day type onions that have low pungency. Some of the commonly planted long day type yellow onion cultivars are Daytona, Ranchero, Granero, Sabroso, Tamara, Hamlet, Fortress, Norstar, Teton, and Vaquero. FIG. 1 is a chart providing measurements of various pungency and sweetness parameters for a number of commercially available (and a few developmental) long day length onion varieties. As will be apparent, a typical long day type onion coming out of storage during the winter months will have a PAD in the 6.2-12 range, and will be quite pungent.

Intermediate day length type onion varieties require 12-14 hours of sunlight before they begin the bulbing process. Short day length type onions are better suited for lower latitudes, as they start the bulbing process when the day-length is only 10-12 hours. In the United States short day type onions are planted primarily in the south during the winter or early spring months. Most sweet or low pungency onions are short day onions, though some intermediate day onions also have low pungency.

Short day varieties do not keep well in storage conditions, and the pungency of short day varieties can climb considerably during storage. Present production in North America and Europe allows harvest of short day onions from mild winter regions from November through April. Long day onions are available fresh in the late summer and as storage onions from September through March, or even year round, have not been available in low pungency varieties. Sweet onions must be imported from the southern hemisphere to fill the gap in sweet onion production (November-February). In the United States, regions like Georgia and Texas produce short day onions from March to June, while low pungency onions available from November to February are short day onions, produced in the southern hemisphere.

Recent years have seen efforts to breed new varieties of low pungency onions and to extend low pungency onion production to new growing areas. Growers in Vidalia, Georgia, use modified atmosphere storage to extend the marketing season of their onions. These and other short day onions have notoriously short storage lives with traditional onion storage practices. Producers in Peru, Chile, Uruguay, Nicaragua, and Mexico produce low pungency SD onion varieties to export to the US during the November to February period, in advance of the first US low pungency onions from Georgia, South Texas, or the Imperial Valley in March or April. Generally, in tropical areas that produce winter onions, short day onions are adapted. In areas that plant in late winter for early summer harvest, intermediate day onions are best. In areas that are too cold to plant until late spring, long day type varieties of onions remain the only option, though no low pungency varieties are available.

Onions marketed as low pungency onions are short day onion varieties, characterized by relatively low solids and high moisture content. Intermediate day types are increasingly being looked to for extending the production and marketing season of low pungency onions over a longer period. Intermediate day types are expected to show intermediate pungency between the sweeter short day onions and the more pungent long day types, as they are crossses between long day and short day types and generally show an intermediate level of pungency.

### SUMMARY OF THE INVENTION

The present invention provides a low pungency, long day type (LD) onion plant, onion seeds and onion bulbs (onions) produced by the plant. The terms “onions”, “bulbs” and “onion bulbs” are used throughout to refer to the harvestable and commercial portion of the onion plant. In one embodiment, the onion produces a white, red or yellow onion bulbs. In another embodiment, the onion produces Spanish type onions.

The LD onion plant produces mature onions that are low pungency, i.e., having a mean PAD measurement after storage of less than 5.5 μM/g FW of pyruvate. In one preferred embodiment, the mean PAD measurement after two months storage is less than 5.0 μM/g FW of pyruvate, though in other preferred embodiments, the mean PAD measurement after two months storage is less than 4.5 μM/g FW of pyruvate more preferably, less than 4.0 μM/g FW of pyruvate, even more preferably, less than about 3.75 μM/g FW of pyruvate.

The invention further provides a LD onion plant having the trait of producing mature onions that are low pungency after storage, where the onions remain at a low pungency level at least two months under normal storage conditions, preferably at least about four months under storage conditions, while in further preferred embodiments, the onions remain at a low pungency level at least about six months under storage conditions.

In one aspect of the invention, mature onions harvested from the onion plant have a mean PAD measurement after two months storage that is about equal to or less than the PAD measurement after two months storage for line WYL 77-5128B, when grown under comparable field conditions.

In another aspect of the invention, onions harvested from the onion plant have a mean PAD measurement after two months storage that is about equal to or less than the PAD measurement at harvest for line WYL 77-5168B, when grown under comparable field conditions.

The invention also provides LD onions harvested from a low pungency, LD onion, for instance, onions harvested from a plurality of low pungency, LD onion plants grown in a field of onion plants.

The invention further provides a method of producing an onion crop comprising growing a plurality of low pungency, LD onion plants and harvesting LD onions from the onion plants. Long day onions harvested according to this method are also provided.

Seed of the inbred LD onion (Allium cepa) plants designated WYL 77-5128B have been deposited at the National Collections of Industrial and Marine Bacteria (NCIMB Limited, Ferguson Building, Craibstone Estate, Bucksburn, Aberdeen, Scotland; AB219YA, UK) as accession No. 41329. Seed of the inbred LD onion plants designated WYL 77-5168B have similarly been deposited at NCIMB, as accession No. 41330. The seed, plants grown from the seed, and seed derived from such plants, are also provided by this invention.

In a further aspect of the invention, seed is provided for a LD onion plant having as at least one parent a plant grown from seed of any one of LD onion plants WYL 77-5128B and WYL 77-5168B. The plant may be a hybrid plant having one or both parents selected from the LD onion plants WYL 77-5128B and WYL 77-5168B. The invention further provides a LD onion plant, or parts thereof, produced by growing seed of such a LD onion plant.

The invention also provides LD onions harvested from a LD onion plant grown from the LD onion plant produced from such seed or by any means of asexual reproduction.

The invention also provides long day onion plants having the physiological and morphological characteristics of the LD onion plants WYL 77-5128B and WYL 77-5168B. The invention contemplates plants or plant products produced from protoplasts or regenerable cells from the LD onion plants, using tissue culture where the cells or protoplasts are produced from a plant tissue selected from the group consisting of: leaf, pollen, cotyledon, hypocotyl, embryos, root, pod, flower, shoot and stem.

Another aspect of the invention provides low pungency, LD onion plants or parts thereof, where the plant or parts thereof have been transformed to contain one or more transgenes operably linked to regulatory elements functional in the LD onion plant.

A still further aspect of the invention provides pollen or an ovule of WYL 77-5128B and WYL 77-5168B, or an onion tissue culture derived from cells of such LD onion plants, particularly a LD onion plant regenerated from such tissue culture and having the trait of being low pungency, long day length.

Use of onion plants or any parts thereof of the invention, such as for breeding purposes, is also provided by the invention. More particularly, a method is contemplated whereby an F1 onion plant is grown from F1 seed resulting from a cross of a LD, low pungency onion plant of the invention as at least one parent onion plant, and selecting progeny onion plants having desired traits. In a further preferred embodiment, two or more generations of back crossing to one of the parent onion plants is used in breeding a new line of LD, low pungency onion plant.

These and other features and advantages of this invention are described in, or are apparent from, the following detailed description of various exemplary embodiments of the devices and methods according to this invention.

### BRIEF DESCRIPTION OF THE DRAWINGS

Various exemplary embodiments of this invention will be described in detail, with reference to the following figures, wherein:

FIG. 1 is a chart showing the results of measurements of various pungency and sweetness levels for a number of commercial varieties.

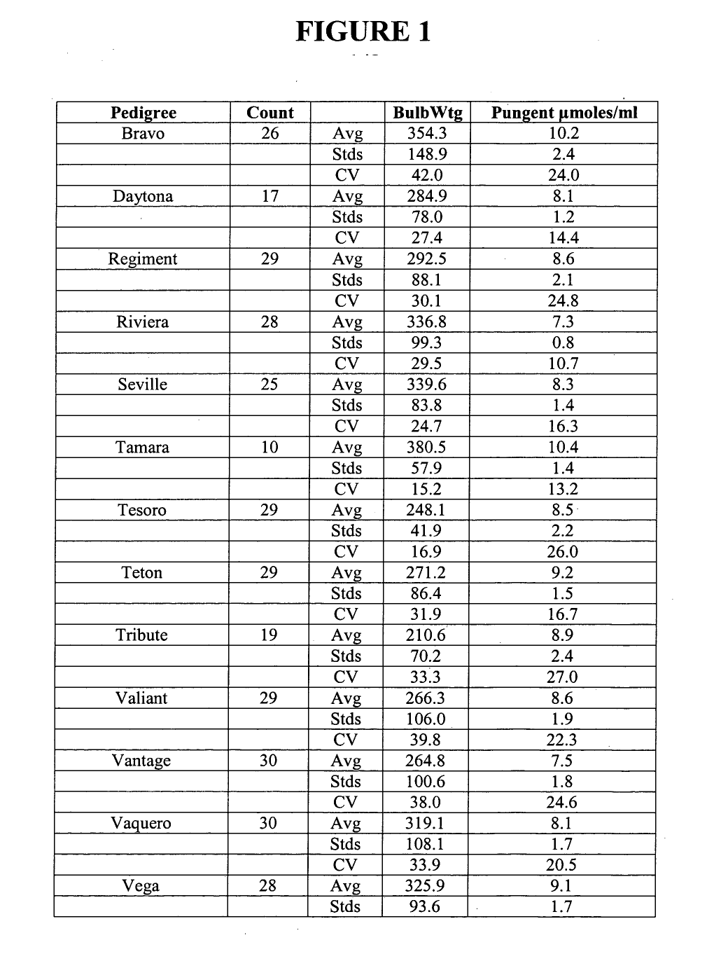

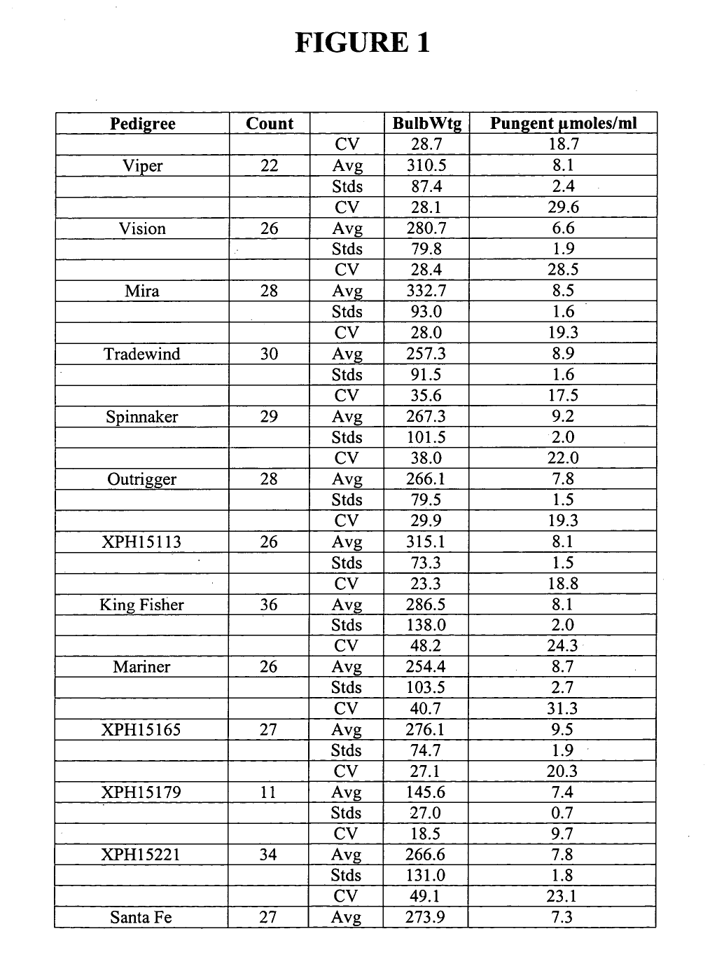

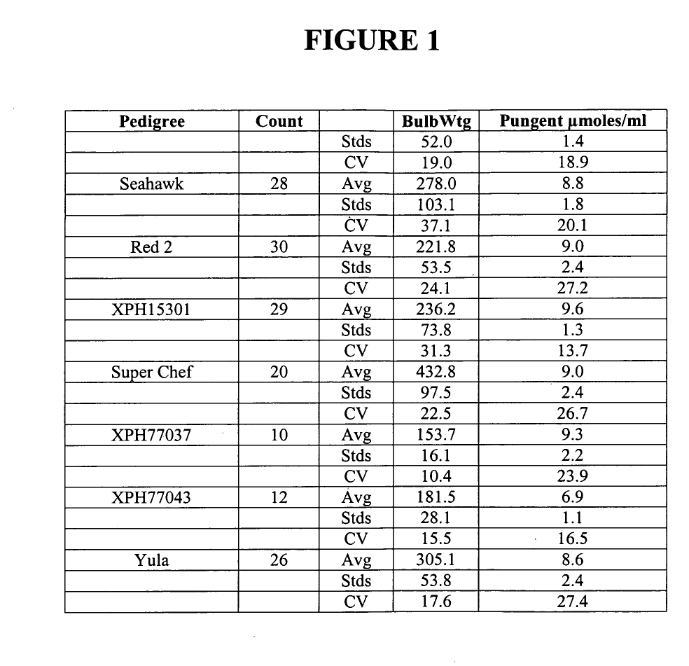

### DETAILED DESCRIPTION OF THE PREFERRED EMBODIMENTS

Onion plants of the present invention are a variety of Allium cepa with improved traits for flavor, in particular, LD onion plants producing LD onions having low pungency.

Definitions

Onion Characteristics

“Onion”, Allium cepa L. (common onion) is a cool season (tolerant of frost) biennial plant. By “biennial plant” it is meant that Allium cepa L. produces a bulb in the first season and seeds in the second. Optimum temperatures for growth and development for most varieties are between 55 and 75° F. However, optimum temperatures for seedling growth are much narrower, with 68 to 77° F. being most productive.

Onions, Onion Bulbs, Bulb

By “bulb” or “onion bulb” is meant the commercially harvested, edible portion of the onion plant. The bulb is comprised of concentric, enlarged fleshy leaf bases, also called scales. At maturity, the outer leaf base dries and becomes scaly as the inner leaf bases thicken and develop into a harvestable bulb.

Pungency

Vidalia Labs International, Inc. has established the short day industry standards for pungency, by measure of mean PAD levels. By the established standards, “low pungency” means onions having a PAD of less than 5.5 μM/g FW of pyruvate. Low pungency onions are also referred to as “sweet” or “mild”. “Medium pungency” onions have a PAD of 5.5 to 6.2 μM/g FW of pyruvate. “High pungency” onions have a PAD of greater than 6.2 μM/g FW of pyruvate.

Day Length and Onion Plants

Onion breeders have three categories of onions according to their day length requirements for bulb initiation. A “short day” length type onion plant (short day, or SD, onion) responds to 11 to 12 hours of daylight; an “intermediate day” length type onion plant (intermediate day, or ID, onion) needs 12 to 14 hours of daylight; and a “long day” length type onion plant (long day, or LD, onion) requires 14 or more hours of daylight for bulb formation to start.

Pyruvic Acid Development (PAD)

Pyruvic acid development measurements, or PAD, are given in micromoles of measured enzymatic pyruvic acid per gram fresh weight of onion flesh (μM/g FW).

Similar Field Conditions

Climate and soils affect pyruvic acid concentrations. Low pungency onions should be grown on soils which have a naturally low sulphur level. High summer temperatures during the growth period are known to increase pyruvic acid concentrations. When producing low pungency onions the use of those sowing and harvesting conditions should be observed that avoid the hottest periods in individual growing regions. Planting density also affects onion pungency. Low plant populations are recommended for low pungency onions. As used herein, then, “similar field conditions” refers to appropriate onion growing conditions for purposes of comparing low pungency lines, i.e., growth in the same field and season, in both a region and under conditions appropriate for producing low pungency onions.

Storage Conditions

Storage conditions for long-term storage of onion bulbs from zero to about six months are under controlled climate conditions of about 4 degrees centigrade to about 6 degrees centigrade and relative humidity in the range from about 50% to about 65%. For long-term storage, onions are harvested when fully mature (leaves are flattened and withered) and typically allowed to dry in the sun. The bulbs are turned, particularly after rainy or damp weather, and damaged material rejected. Containers providing air circulation are used, including such things as shallow slatted trays and open mesh sacks. Exposure to light is avoided, because light can induce sprouting, and the onions are kept dry. When handled under such conditions, a long storage life onion can be successfully stored for about six or seven months, with losses of less than 25%.

Onion Breeding

Historically, onion populations were maintained by open pollination, with considerable phenotypic variation for such traits as size, shape, pungency and color. The production of hybrid varieties is increasingly favored, however, such that commercial onion cultivars are today predominantly hybrids. Male sterile onion lines are becoming available for integration into commercial breeding programs. The production of hybrid varieties requires homozygous inbred parental lines and, preferably, the availability of reversible male sterility. Complete homozygosity of the inbred, or parental, lines ensures uniformity of the F1 hybrid crops, while male sterility facilitates large-scale hybrid seed production.

The use of male sterile inbreds is but one factor in the production of onion hybrids. The development of onion hybrids in a onion plant breeding program requires, in general, the development of homozygous inbred lines, the crossing of these lines, and the evaluation of the crosses. Onion plant breeding programs combine the genetic backgrounds from two or more inbred lines or various other germplasm sources into breeding populations from which new inbred lines are developed by selfing and selection of desired phenotypes. Hybrids also can be used as a source of plant breeding material or as source populations from which to develop or derive new onion lines. Plant breeding techniques known in the art and used in an onion plant breeding program include, but are not limited to, recurrent selection, backcrossing, double haploids, pedigree breeding, restriction fragment length polymorphism enhanced selection, genetic marker enhanced selection, and transformation. Often a combination of these techniques are used. Thus, inbred lines derived from hybrids can be developed using plant breeding techniques as described above. New inbreds are crossed with other inbred lines and the hybrids from these crosses are evaluated to determine which of those have commercial potential.

Backcrossing methods can be used with the present invention to improve or introduce a particular characteristic or set of characteristics into an inbred. The term “backcrossing” as used herein refers to the repeated crossing of a hybrid progeny back to one of the parental onion plants for that inbred. The parental onion plant which contributes the gene for the desired characteristic is termed the nonrecurrent, or donor, parent. This terminology refers to the fact that the nonrecurrent parent is used one time in the backcross protocol and therefore does not recur. The parental onion plant to which the gene or genes from the nonrecurrent parent are transferred is known as the recurrent parent, as it is used for several rounds in the backcrossing protocol. In a typical backcross protocol, the original inbred of interest (recurrent parent) is crossed to a second inbred (nonrecurrent parent) that carries the trait or traits of interest to be transferred. The resulting progeny from this cross are then crossed again to the recurrent parent and the process is repeated until a onion plant is obtained wherein essentially all of the desired morphological and physiological characteristics of the recurrent parent are recovered in the converted plant, in addition to the one or few transferred traits of the nonrecurrent parent. Typically, four or more backcross generations will be required, with selection for the desired trait, before the progeny will contain essentially all genes of the recurrent parent except for the genes controlling the desired trait(s). Where molecular markers are available for use during the selection process, the program may be accelerated. The last backcross generation is then selfed to give pure breeding progeny for the gene(s) being transferred.

When the term “inbred”, “inbred plant” or “inbred onion” is used in the context of the present invention, this also includes any single gene conversions of that inbred. The term single gene converted plant as used herein refers to those onion plants which are developed by a plant breeding technique called backcrossing wherein essentially all of the desired morphological and physiological characteristics of an inbred are recovered in addition to the single gene transferred into the inbred via the backcrossing technique.

Low Pungency, LD Onions

A low pungency, LD onion is a long day length type onion plant that will produce “sweet” or “low pungency” mature onion bulbs having a mean PAD value of less than about 5.5 μM/g FW, when stored for two months, four months or six months under long-term storage conditions. A low pungency, LD onion will also have a mean PAD value that is equal to or less, when grown under those comparable field conditions, than the mean PAD values for onions of the long day length line WYL 77-5168B, under long-term storage conditions for two months, four months or six months. Alternatively, a low pungency, LD onion will have a mean PAD value that is equal to or less, when grown under those comparable field conditions, than the mean PAD values for onions of the long day length line WYL 77-5128B, under long-term storage conditions for two months, four months or six months.

As described herein, onion plants are provided that produce sweet or low pungency onion bulbs under long day conditions. The low pungency, LD onions arose from an accidental discovery of two lines that are in the LD class for bulb production, yet which do not produce high pungency with the increased sunlight of long day growing conditions.

With the present invention, the grower is able to produce a low pungency onion that can be grown under long day conditions, and which stores well. The trait of bulb production under long day conditions and low pungency line is a great benefit to the grower, as it expands the areas where the desirable low pungency onions may be produced.

Unlike short day onions, the low pungency, LD onions will store for two months or longer periods under long term storage conditions, and hold their low pungency. Because of the long storage capabilities of the low pungency, LD onions, they can fill a gap in the present production of low pungency onions (November-February). The low pungency, LD onions may be stored for two months up to six months, providing a single, continuous source of low pungency onions from growing regions in northern latitudes.

WYL 77-5168B is LD Spanish onion breeding line, combining all the desired features of typical LD Spanish onions, only with low pungency. The variety that most closely resembles is WYL 77-5168B is Vision. The comparative characteristic that most readily distinguishes the low pungency varieties such as WYL77-5168B from all other LD varieties is the unique low pungency (“mildness”) of WYL 77-5168B. This level of low pungency is unique for onions that respond to photoperiod under long day conditions to induce bulbing.

Seed from Allium cepa breeding line WYL 77-5168B, described above, was deposited on 24 Jun. 2005 with NCIMB Ltd., as Accession No. NCIMB 41330 Allium cepa WYL 77-5168B.

WYL 77-5128B is LD Spanish onion breeding line, combining all the desired features of typical LD Spanish onions with low pungency. Again, the variety that most closely resembles WYL 77-5128B is Vision. WYL 77-5128B produces a low pungency onion under photoperiods of long day conditions to induce bulbing. Seed from Allium cepa breeding line WYL 77-5128B, described above, was deposited on 24 Jun. 2005 with NCIMB Ltd., as Accession No. NCIMB 41329 Allium cepa WYL 77-5128B.

### EXAMPLES

WYL 77-5128B and WYL 77-5168B are LD onions producing bulbs under long day conditions. Various field trials were conducted with breeding lines in selecting low pungency, LD onions, comparing the low pungency, LD onions with control LD onions, including Vision, grown under comparable field conditions. Vision is a CMS hybrid presently marketed by Seminis, a hybrid yellow Spanish type onion of LD class, with full season maturity and excellent storage.

Collected onion samples were analyzed for pyruvate (mean PAD levels), according to the methods described (Schwimmer, S.; Weston, W. 1961. Onion Flavor and Odor, Enzymatic Development of Pyruvic Acid in Onions A Measure Of Pungency. Journal Of Agricultural And Food Chemistry 9:301).

As is true with other onion cultivars, a small percentage of variants can occur within commercially acceptable limits for almost any characteristics during the course of onion multiplication. No variants were observed during the years in which WYL 77-5128B and WYL 77-5168B were observed to be uniform and stable.

### Example 1

### WYL 77-5168B, Long Day Onion

WYL 77-5168B, a LD onion inbred, was developed by mass selection from a synthetic gene pool of Yellow Sweet Spanish onion (synthetic gene pool YSS-715B). Mass selection is the formation of a composite population through selective harvest of individuals among a heterozygous population. See, Burton, G. W. 1990. Enhancing germplasm with mass selection. p. 99-100. In: J. Janick and J. E. Simon (eds.), Advances in new crops. Timber Press, Portland, Oreg.

Synthetic gene pool YSS-715B was developed from a pool of YSS 53-351B (50%), and YSS 805 (50%). This pool was created for other phenotypic purposes, with no initial interest with regard to pungency.

WYL 77-5168B is a Spanish onion line that is large in size, with a round bulb shape and medium colored skin. Tops are medium and somewhat floppy. Storage length of WYL 77-5168B is medium term. Maturity of WYL 77-5168B is full-season for a Spanish type onion.

1994 Mass 1 generation bulbs were selected from synthetic YSS-715B.

1995 Seed was produced on the M1 bulb selection.

1996 Mass 2 generation bulbs were selected from 1995 seed production.

1997 Seed was produced in cage 97-575 from the 1996 M2 bulb selection.

1998 Mass 3 generation bulbs were selected from source 97-575.

1999 Seed was produced from M3 bulb selections in cage 99-327-1. Additional bulbs were also grown of source 97-575 in row number 99-7099 and bulb selections were made.

2000 Mass 4 generation seed was produced in cage 00333-1 from bulbs grown in row number 99-7099.

2001 Bulbs were grown in row 01OS3164 from original seed source 00333-1 and parent breeding line WYL 77-5168B was assigned. WYL 77-5168B showed good characteristics for size, skin retention and lack of bolters (seed-stem formation, or “bolting”, produces poor quality bulbs with a hard center making them unmarketable). This line also produced sufficient seed for further development. In the initial screen, WYL 77-5168B had a mean PAD of 4.53 μM/g FW. WYL 77-5128B had a mean PAD of 6.48 μM/g FW. Three other lines, less favorable for other traits, had mean PADs of 5.82, 6.16 and 6.22 μM/g FW. Of the remaining lines tested, there were ten between 6.5 and 7.0, twenty-one between 7.0 and 7.5, eleven between 7.5 and 8.0, fourteen between 8.0 and 8.5, fourteen between 8.5 and 9.0, ten between 9.0 and 9.5, three between 9.5 and 10.0, eight between 10.0 and 10.5, and five greater than 10.5. The low PADs for certain of the lines led to selection for those lines in subsequent generations based on mean PAD levels after two months storage under long term storage conditions.

2002 Mass 5 generation seed was produced in cage RNG4005 from bulbs grown in row number 01OS3164. Additional bulbs were grown in row 02OS3097 from seed source 00333-1. Pungency testing on these bulbs showed line WYL 77-5168B having pungency level of 4.53 μM/g FW PAD (min 2.14, max 6.98) after two months of storage.

2003 Seed Production Cycle—Mass 5 generation seed was produced in cages RNR5613, RNR5616, RNR5617, RNR5619 from bulbs grown in row number 02OS3097 from original source 00333-1 (M-4). Pungency testing on these bulbs showed line WYL 77-5168B having a pungency level of 4.51 PAD (min 2.30, max 9.97) after six months of storage. Cage RNR5619 was a low pungency selection with a mean of 3.91 PAD (min 2.56, max 4.96).

2003 Bulb Production Cycle—Bulbs of WYL 77-5168B were grown in bulb row 03OS4045 from original seed source 00333-1. Pungency testing on bulb row 03OS4045 showed line WYL 77-5168B having a mean pungency of 5.16 PAD (min 1.82, max 11.94) after six months of storage. Vision was grown in rows 03OS1283 and 03OS1317 and pooled for analysis and had a mean pungency level of 9.15 PAD (min 6.25, max 15.44) after six months of storage.

2004 Seed Production Cycle—Seed was produced of line WYL 77-5168B in cage RNV4012 from original seed source 00333-1. RNV4012 was a low pungency selection with a mean level of 3.74 PAD (min 1.82, max 4.57).

2004 Bulb Production Cycle—The bulbs of WYL 77-5168B from an original source 00333-01 were tested after two months of storage and showed a mean pungency level of 6.02 PAD (min 4.28, max 8.19). An advanced selection of WYL 77-5168B, RNR5616-2 was grown in row 04OS7076 showed a mean pungency level of 4.09 PAD (min 3.52, max 5.75) after two months of storage. An advanced selection of WYL 77-5168B, RNR5616-12 was grown in row 04OS7085 showed a mean pungency level of 4.87 PAD (min 3.80, max 7.45) after two months of storage. An advanced selection of WYL 77-5168B, RNR5616-7 was grown in row 04OS7081 showed a mean pungency level of 5.09 PAD (min 3.68, max 6.75) after two months of storage. An advanced selection of WYL 77-5168B grown in row 04OS7086, RNR5616-15 showed a mean pungency level of 5.15 PAD (min 3.86, max 6.08) after two months of storage. An advanced selection of WYL 77-5168B, RNR5616-3 was grown in row 04OS7077 showed a mean pungency level of 5.30 PAD (min 3.70, max 5.75) after two months of storage. Vision was also analyzed and from replication 1 showed a mean pungency level of 6.07 PAD (min 4.94, max 7.24) and from replication 2 showed a mean pungency level of 6.25 (min 5.14, max 7.40). Peruvian Sweets (Bland Farms source), short day type (SD) onions imported and purchased for this study, showed a mean pyruvate level of 6.04 PAD (min 4.67, max 7.94).

Selection criteria in the field represent a balance of characteristics related to productivity and fit to the market including, yield potential, foliage, bulb shape, bulb skin, bolting tolerance, long storage, and resistance to pink root and fusarium.

Observations in 2001, 2002, 2003, and 2004 confirm that breeding line WYL 77-5168B is uniform and stable within commercially acceptable limits.

### Example 2

### WYL 77-5128B, Long Day Onion

WYL 77-5128B, a LD Spanish onion inbred, was developed by mass selection from a synthetic gene pool of Yellow Sweet Spanish (synthetic gene pool YSS-FR713B).

Synthetic gene pool YSS-FR713B was developed from a pool of YSS-Giano (50%), YSS 53-351B (25%), and YSS Peckham (25%).

WYL 77-5128B is a low pungency Spanish onion line, that is medium in size, with a high globe shape and dark colored skin. Tops are large and upright. Storage length of WYL 77-5128B is long term. Maturity of WYL 77-5128B is mid-season for a Spanish type onion

1995 Mass 1 generation bulbs were selected from synthetic YSS—FR713B.

1996 Seed was produced on the M1 bulb selection.

1997 Mass 2 generation bulbs were selected from 1996 seed production.

1998 Seed was produced in cage 97-547 from the 1996 M2 bulb selection.

1999 Mass 3 generation bulbs were selected from source 97-547.

2000 Seed was produced from M3 bulb selections in cage 00335-1.

2001 Mass 4 generation bulbs were selected in row 01OS3182 (from original seed source 00335-1) and parent breeding line WYL 77-5128B was assigned. As noted above, selections from this point forward were screened for mean PAD levels after two months storage under long term storage conditions.

2002 Seed was produced from 01OS3182 bulb production in cages RNG3243 and RNG3968 (M4 seed). Bulbs were grown in row 02OS3029 from original seed source 00335-1. Pungency testing on these bulbs showed line WYL 77-5128B having pungency level of 5.40 PAD (min 3.41, max 8.23) after two months of storage.

2003 Seed production of Mass 4 generation seed was produced in cage RNR5724 and RNR5621 from bulbs grown in row number 02OS3029. Cage RNR5621 was a low pungency selection with a mean of 4.72 (min 3.41, max 5.58).

2003 Bulb production Cycle—Bulbs were grown in row 03OS4043 from seed source 00335-1. Pungency testing on bulb row 03OS4043 showed line WYL77-5128B having a mean pungency of 7.87 PAD (min 4.38, max 11.93) after six months of storage. Vision was grown in rows 03OS1283 and 03OS1317 and pooled for analysis and had a mean pungency level of 9.15 PAD (min 6.25, max 15.44) after six months of storage. This trial received hail damage and did not develop normal size, resulting in smaller more pungent bulbs.

2004 Seed was produced of line WYL 77-5128B in cage RNV4014 from seed source 00335-1. RNV4014 was a low pungency selection with a mean level of 5.96 PAD (min 4.38, max 6.50).

2004 Bulb production cycle—The bulbs of selection WYL 77-5128B from original seed source 00335-1 were tested after two months of storage and showed a mean pungency level of 5.32 PAD (min 3.24, max 6.95). An advanced selection of WYL 77-5128B, RNR5621-13 was grown in row 04OS7097 showed a mean pungency level of 4.29 PAD (min 3.12, max 5.60) after two months of storage. An advanced selection of WYL 77-5128B, RNR5621-15 was grown in row 04OS7099 showed a mean pungency level of 4.37 PAD (min 3.40, max 5.59) after two months of storage. An advanced selection of WYL 77-5128B, RNR5621-5 was grown in row 04OS7090 showed a mean pungency level of 4.53 PAD (min 3.32, max 6.20) after two months of storage. An advanced selection of WYL 77-5128B, RNR5621-8 was grown in row 04OS7093 showed a mean pungency level of 4.67 PAD (min 3.26, max 5.60) after two months of storage. An advanced selection of WYL 77-5128B, RNR5621-12 was grown in row 04OS7096 showed a mean pungency level of 4.81 PAD (min 3.88, max 6.49) after two months of storage. An advanced selection of WYL 77-5128B, RNR5621-10 was grown in row 04OS7095 showed a mean pungency level of 4.87 PAD (min 3.94, max 5.75) after two months of storage. An advanced selection of WYL 77-5128B, RNR5621-3 was grown in row 04OS7089 showed a mean pungency level of 5.03 PAD (min 3.82, max 6.01) after two months of storage. An advanced selection of WYL 77-5128B, RNR5621 was grown in row 04OS7009 showed a mean pungency level of 5.10 PAD (min 3.65, max 8.79) after two months of storage. Vision was also analyzed and from replication 1 showed a mean pungency level of 6.07 PAD (min 4.94, max 7.24) and from replication 2 showed a mean pungency level of 6.25 (min 5.14, max 7.40). Peruvian Sweets (Bland Farms source), SD onions imported and purchased for this study, showed a mean pyruvate level of 6.04 PAD (min 4.67, max 7.94).

Selection criteria in the field represent a balance of characteristics related to productivity and fit to the market including, yield potential, foliage, bulb shape, bulb skin, bolting tolerance, long storage, and resistance to pink root and fusarium.

### Example 3

### Pungency in Storage

Harvested low pungency long day onions were stored under storage conditions of 5 degrees centigrade and 50% relative humidity for 6 months.

Tables 1 and 2 below provide raw data for mean PAD measurements after two and six months storage under long term storage conditions on the lines noted above.

TABLE 1
History of pungency selection of line WYL 77-5168B
Mean PAD

| | 2002 | 2003 | 2004 |
|---|---|---|---|
| Source | Bulb Harvest | Bulb Harvest | Bulb Harvest |
| 00333-1 | 4.53 g | 4.51 g FW (2)(3) | 6.02 g FW (1) |
| (original source) | FW (2) | | 4.20 g FW (2) |
| RNR5619 | | | 5.60 g FW (1) |
| (selection of 00333-1) | | | 4.16 g FW (2) |
| RNR5616-2 | | | 4.09 g FW (1) |
| (selection of 00333-1) | | | 4.98 g FW (2) |
| RNR5616-3 | | | 5.30 g FW (1) |
| (selection of 00333-1) | | | 4.24 g FW (2) |
| RNR 5616-7 | | | 5.09 g FW (1) |
| (selection of 00333-1) | | | 4.12 g FW (2) |
| RNR 5616-12 | | | 4.87 g FW (1) |
| (selection of 00333-1) | | | 4.91 g FW (2) |
| RNR 5616-15 | | | 5.15 g FW (1) |
| (selection of 00333-1) | | | 3.28 g FW (2) |
| Vidalia Sweets | | 9.15 g FW (4) | 3.34 g FW (4) |
| (SD) | | | |
| Vision | | 9.15 g FW (3) | 6.16 g FW (1) |
| (LD Spanish Variety) | | | 8.71 g FW (2) |
| TX 1015Y | | | 4.69 g FW (4) |
| (SD control variety) | | | |
| Peruvian Sweet | | | 6.02 g FW (4) |
| (SD) | | | |

(1) After two months storage
(2) After six months storage
(3) Field received damage from a hail storm prior to harvest, which reduced size and stopped normal maturity.
(4) Locally purchased source

TABLE 2
History of pungency selection of line WYL 77-5128B
Mean PAD

| | 2002 | 2003 | 2004 |
|---|---|---|---|
| Source | Bulb Harvest | Bulb Harvest | Bulb Harvest |
| 00335-1 | 5.40 g | 7.87 g FW (2)(3) | 5.32 g FW (1) |
| (original source) | FW (2) | | 6.30 g FW (2) |
| RNR 5621 | | | 5.10 g FW (1) |
| (selection of 00335-1) | | | 6.21 g FW (2) |
| RNR 5621-3 | | | 5.06 g FW (1) |
| (selection of 00335-1) | | | 5.84 g FW (2) |
| RNR 5621-5 | | | 4.53 g FW (1) |
| (selection of 00335-1) | | | 6.35 g FW (2) |
| RNR 5621-8 | | | 4.67 g FW (1) |
| (selection of 00335-1) | | | 6.01 g FW (2) |
| RNR 5621-10 | | | 4.87 g FW (1) |
| (selection of 00335-1) | | | 5.75 g FW (2) |
| RNR 5621-12 | | | 4.81 g FW (1) |
| (selection of 00335-1) | | | 6.00 g FW (2) |
| RNR 5621-13 | | | 4.29 g FW (1) |
| (selection of 00335-1) | | | 5.99 g FW (2) |
| RNR5621-15 | | | 4.37 g FW (1) |
| (selection of 00335-1) | | | 5.90 g FW (2) |
| Vidalia Sweets | | 9.15 g FW (4) | 3.34 g FW (4) |
| (SD) | | | |
| Vision | | 9.15 g FW (3) | 6.16 g FW (1) |
| (LD Spanish Variety) | | | 8.71 g FW (2) |
| TX 1015Y | | | 4.69 g FW (4) |
| (SD control variety) | | | |
| Peruvian Sweet | | | 6.02 g FW (4) |
| (SD) | | | |

(1) After two months storage
(2) After six months storage
(3) Field received damage from a hail storm prior to harvest, which reduced size and stopped normal maturity and made much smaller bulbs.
(4) Locally purchased source

### Example 4

### Long Day Length Type, Storage Onions

Long day length type, low pungency onions were stored under storage conditions of 5 degrees centigrade and 50% relative humidity for 6 months and evaluated for marketable yield. Marketable yield, sometimes called “storability”, refers to the retention of onion quality at a high level. Quality is assessed by monitoring internal sprout development, as well as the presence of surface mold and decay.

Table 3 provides data for storability, as percentage marketable bulbs remaining after six months storage under long term storage conditions. The B line designation refers to a more advanced line, having completed several generations of selection.

TABLE 3
Storage Characteristics of WYL 77-5128B and WYL 77-5168B
% marketable bulbs

| | Storage % | Storage % | Storage % | Storage % | Storage % |
|---|---|---|---|---|---|
| | after 2 | after 3 | after 4 | after 5 | after 6 |
| | months | months | months | months | months |
| WYL 77- | 98.8% | 97.6% | 96.4% | 96.4% | 95.2% |
| 5128B | | | | | |
| (LD type) | | | | | |
| WYL 77- | 98.7% | 97.4% | 97.4% | 82% | 74.3% |
| 5168B | | | | | |
| (LD type) | | | | | |
| Vision | 100.0% | 98.6% | 93.1% | 93.1% | 87.7% |
| (LD type) | | | | | |
| Peruvian | 79.7% | 72.4% | 53.6% | 37.7% | 11.6% |
| Sweets | | | | | |
| (SD type) | | | | | |

The results in Table 3 demonstrate that the LD, low pungency onions provide excellent storage up to 6 months.

### Example 5

### Plant Breeding and Line Development

Various Allium cepa lines of this invention can be used to transmit the long day photoperiodic response, low pungency trait to new varieties using various cross pollination and selection methods. Therefore, breeders may obtain hybrids using the described low pungency, LD onion plants and lines for further selfing and subsequent selection. Using standard crossing, backcrossing and selection techniques, those of skill in the art may obtain commercial low pungency, LD onions with various desirable traits besides those described above. For example, breeders may easily obtain commercial Allium cepa lines with the preferred trait of LD onion color, disease resistance traits, traits relating to optimized yield under specific growth conditions and other agronomic or consumer preferred characteristics.

### Example 6

### Onion Production

Onions are usually planted in multiple rows on beds. Beds are commonly formed at or just before planting with 2 to 12 rows planted per bed. A typical arrangement is two double rows spaced about 12 inches apart on 34- or 44-inch beds. Multiple such arrangements are sometimes used, particularly with drip irrigation systems. Some low pungency onions are planted in single rows.

Onion seed is expensive, and is generally direct seeded with precision planters (onions are not thinned). Seed is commonly planted about ¼- to ½-inch deep. With furrow irrigation in lighter textured soils or with limited soil moisture, a depth of ¾ inch may be required.

Hybrid seed of low pungency, LD onions are planted in the conventional way and onions are grown to maturity, harvested, and maintained under long-term storage conditions for a period of two to six months. Low pungency onions are thereafter removed from storage and provided during the winter to the consumer as a sweet onion.

Although the foregoing invention has been described in some detail by way of illustration and example for purposes of clarity and understanding, it will be obvious that certain changes and modifications may be practiced within the scope of the invention, as limited only by the scope of the appended claims.

All references cited herein are hereby expressly incorporated herein by reference.

### DEPOSIT INFORMATION

A deposit has been made of the Seminis Vegetable Seeds proprietary inbred Allium cepa lines disclosed above and recited in the appended claims, with NCIMB Ltd, 23 St. Machar Drive, Aberdeen AB24 3RY, an International Depositary Authority (IDA) as established under the Budapest Treaty on the International Recognition of the Deposit of Micro-organisms for the Purposes of Patent Procedure. The NCIMB accession numbers for lines WYL 77-5128B and WYL 77-5168B are, respectively, NCIMB 41329 and NCIMB 41330. The date of each of the deposits was 24 Jun. 2005.

Upon issuance of a patent, all restrictions upon the deposit will be removed, and the deposit is intended to meet all of the requirements of 37 C.F.R. § 1.801-1.809.

What is claimed is:

1. A low pungency, long day type onion plant.

2. The onion plant of claim 1, wherein the plant is a yellow onion.

3. The onion plant of claim 1, wherein the plant is a Spanish onion.

4. The onion plant of claim 1, wherein the plant produces onions having a mean PAD measurement at harvest of less than 5.5 μM/g FW.

5. The onion plant of claim 4, wherein the plant produces onions having a mean PAD measurement at harvest of less than 5.0 μM/g FW.

6. The onion plant of claim 5, wherein the plant produces onions having a mean PAD measurement at harvest of less than 4.5 μM/g FW.

7. The onion plant of claim 6, wherein the plant produces onions having a mean PAD measurement at harvest of less than 4.0 μM/g FW.

8. The onion plant of claim 6, wherein the plant produces onions having a mean PAD measurement at harvest of less than 3.75 μM/g FW.

9. The onion plant of claim 1, wherein mature onions harvested from said plant maintain a low pungency level for at least about two months under storage conditions.

10. The onion plant of claim 9, wherein mature onions harvested from said plant maintain a low pungency level for at least about four months under storage conditions.

11. The onion plant of claim 10, wherein mature onions harvested from said plant maintain a low pungency level for at least about six months under storage conditions.

12. The onion plant of claim 1, wherein the pungency of mature onions harvested from said plant is lowered after six months under storage conditions.

13. The onion plant of claim 12, wherein the mean PAD measurement is lowered at least about 10%.

14. The onion plant of claim 1, wherein mature onions harvested from said plant have a mean PAD measurement at harvest that is equal to or less than the PAD measurement at harvest for onions of breeding line WYL 77-5128B, when grown under comparable field conditions.

15. The onion plant of claim 1, wherein mature onions harvested from said plant have a mean PAD measurement at harvest that is equal to or less than the PAD measurement at harvest for onions of breeding line WYL 77-5168B, when grown under comparable field conditions.

16. Onions harvested from an onion plant of claim 1.

17. A plurality of onion plants of claim 1 grown in a field.

18. A method of producing an onion crop comprising growing a plurality of onion plants of claim 1 and harvesting onions from the onion plants.

19. Onions harvested according to the method of claim 18.

20. Seed of an inbred onion plant designated WYL 77-5128B, a sample of the seed having been deposited 24 Jun. 2005 as Accession No. NCIMB 41329 Allium cepa WYL 77-5128B.

21. An onion plant having the physiological and morphological characteristics of the onion plant of claim 20.

22. Seed of an inbred onion plant designated WYL 77-5168B, a sample of the seed having been deposited 24 Jun. 2005 as Accession No. NCIMB 41330 Allium cepa WYL 77-5168B.

23. An onion plant having the physiological and morphological characteristics of the onion plant of claim 22.

24. A method for producing onion seed comprising crossing two parent onion plants and harvesting the resultant onion seed, wherein at least one of said parent onion plants is the onion plant of claim 20 or claim 22.

25. Seed produced by the method of claim 24.

26. An onion plant, or parts thereof, produced by growing seed of claim 25.

27. Onions harvested from an onion plant of claim 26.

28. A tissue culture produced from protoplasts or regenerable cells from the plant of claim 26.

29. The tissue culture according to claim 27, wherein the cells or protoplasts are produced from a plant tissue selected from the group consisting of: leaf, pollen, cotyledon, hypocotyl, embryos, root, pod, flower, shoot and stem.

30. An onion plant regenerated from the tissue culture of claim 29 and having the traits of low pungency and long day length type.

31. The onion plant of claim 30, or parts thereof, wherein the plant or parts thereof have been transformed to contain one or more transgenes operably linked to regulatory elements functional in the onion plant.

32. The method of claim 24, further comprising the step of growing an F1 onion plant from seed resulting from said cross, crossing said F1 onion plant to one of said parent onion plants, and selecting progeny onion plants having desired traits.

33. The method of claim 32 comprising two or more generations of back crossing to one of said parent onion plants.
